(19) United States
(12) Patent Application Publication
(10) Pub. No.: US 2025/0041282 A1
(43) Pub. Date: Feb. 6, 2025

(54) METHOD FOR TREATING INFLAMMATORY PULMONARY DISEASE

(71) Applicant: FU JEN CATHOLIC UNIVERSITY, New Taipei City (TW)

(72) Inventors: Jau-Chen LIN, New Taipei City (TW); Guey-Mei JOW, New Taipei City (TW); Shang-Shing P. CHOU, New Taipei City (TW); Jung-Sen LIU, New Taipei City (TW); Chang-Lin LU, New Taipei City (TW); Fang JUNG, New Taipei City (TW); Shih-Hsing YANG, New Taipei City (TW); Hui-Yun TSENG, New Taipei City (TW)

(21) Appl. No.: 18/696,148

(22) PCT Filed: Sep. 28, 2022
(86) PCT No.: PCT/CN2022/122100

§ 371 Date: Mar. 27, 2024

Related U.S. Application Data
(60) Provisional application No. 63/249,080, filed on Sep. 28, 2021.

(51) Int. Cl. A61K 31/4412; A61P 11/00

(52) U.S. Cl. CPC A61K 31/4412; A61P 11/00

(57) ABSTRACT

Provided is a method for preventing or treating an inflammatory pulmonary disease or disorder in a subject in need thereof, including administering to the subject an effective amount of FJU-C28 or a salt thereof. Also provided is a use of an a compound of FJU-C28 or a salt thereof in the manufacture of a medicament for preventing or treating an inflammatory pulmonary disease or disorder.

### BACKGROUND

### 1. Technical Field

The present disclosure relates to methods for treating an inflammatory pulmonary disease or disorder in a subject in need, and particularly to methods for treating acute respiratory distress syndrome (ARDS) or lung fibrosis.

### 2. Description of Related Art

Despite marked improvements in supportive care, the mortality rate of acute respiratory distress syndrome (ARDS) due to the excessive inflammatory response caused by viral or bacterial infection-induced direct or indirect lung injury is still high. The inflammatory response is an important host defense mechanism that protects against infection and restores damaged tissues to normal physiological states. Macrophages play a crucial role in regulating the innate immune response during inflammatory processes. Lipopolysaccharide (LPS) activates macrophages to release various inflammatory mediators and inflammatory cytokines. However, the prolonged production of inflammatory mediators by macrophages can cause an inflammatory response, eliciting the release of various vascular and cellular danger signals that promote damage to the host and contribute to the pathology of many inflammatory diseases.

Pulmonary fibrosis (PF) is a well-recognized sequela of ARDS. Despite growing knowledge of the pathobiology of PF, the prognosis of patients remains poor. PF is irreversible and currently, there is no therapy to stop or significantly delay the disease progress. Typically, treatment strategies for PF aim to improve quality of life (i.e., relieve disease signs/symptoms) or attempt to limit further inflammation and scarring. Anti-inflammatory drugs, including corticosteroids and cytotoxic agents, are used even though there is no evidence of a benefit for long-term survival. Pirfenidone and Nintedanib are the two FDA approved drugs for the management of IPF. Both Pirfenidone and Nintedanib are reported to reduce fibrotic tissue to some extent in the lungs of patients with pulmonary fibrosis, but the treatment is far from optimal. There is a pressing need for more effective and tolerable next generation therapies or treatments that can significantly delay the progression of pulmonary fibrosis, if not provide a cure and improve patients' overall quality of life (QOL).

Due to the excessive inflammatory response caused by viral or bacterial infection-induced direct or indirect lung injury, the mortality rate of acute respiratory distress syndrome (ARDS) or lung fibrosis is still high. Therefore, there is still an unmet and urgent need in the art to provide new therapeutic approaches for the treatment of these inflammatory pulmonary diseases or disorders.

### SUMMARY

2-Pyridones are a class of potent antibacterial agents that are used to treat bacterial infections caused by gram-negative bacteria; these agents are effective treatments that targets the early release of proinflammatory cytokines and are useful for preventing and/or treating inflammation related to leukocyte infiltration.

The present disclosure is directed to a method for treating acute respiratory distress syndrome (ARDS) or lung fibrosis with a compound named FJU-C28, which is derived from a 2-pyridone compound. In some embodiments of the present disclosure, the anti-inflammatory effects of FJU-C28 on the expression of inflammatory mediators were analyzed in vitro, and the efficacy of FJU-C28 in improving lung function in ALI was evaluated by using an in vivo animal model. In some embodiments, the profile of cytokines in macrophages with LPS-induced inflammation was identified by using a cytokine protein array, and then the molecular mechanism of the dominant cytokines, including IL-6 and RANTES, in the progression to ARDS was manipulated by using in vitro cell models.

In one aspect, the present disclosure provides a method for preventing or treating an inflammatory pulmonary disease or disorder, comprising administering a pharmaceutical composition to a subject in need thereof, wherein the pharmaceutical composition comprises an effective amount of a compound of formula (I) below (i.e., FJU-C28) or a salt thereof:

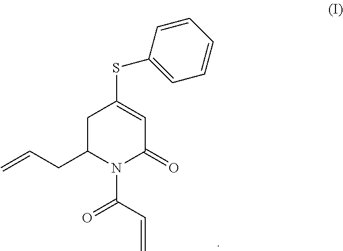 [SMILES: C=CCC1CC(Sc2ccccc2)=CC(=O)N1C(=O)C=C]

In at least one embodiment of the present disclosure, the inflammatory pulmonary disease or disorder is due to the excessive inflammatory response caused by viral or bacterial infection.

In some embodiments, the inflammatory pulmonary disease is acute respiratory distress syndrome or lung fibrosis.

In at least one embodiment of the present disclosure, the compound of formula (I) or a salt thereof is used to suppress mRNA or protein expression of iNOS in the subject.

In at least one embodiment of the present disclosure, the compound of formula (I) or a salt thereof is used to suppress mRNA or protein expression of COX2 in the subject.

In at least one embodiment of the present disclosure, the compound of formula (I) or a salt thereof is used to suppress mRNA or protein expression of a proinflammatory cytokine in the subject. In some embodiments, the proinflammatory cytokine may be RANTES, TIMP1, IL-6, or IL-10. In some embodiments, the proinflammatory cytokine is RANTES or IL-6.

In at least one embodiment of the present disclosure, the effective amount of the compound of formula (I) or a salt thereof is between 0.1 to 10 μM, such as 0.5 to 10 μM, 1 to 5 μM, or 2 to 7 μM. In some embodiments, the effective amount of the compound of formula (I) or a salt thereof is about 0.1 μM, 0.2 μM, 0.5 μM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 9.6 μM, 9.7 μM, 9.8 μM, 9.9 μM, or 10 μM.

In at least one embodiment of the present disclosure, the effective amount of a compound of formula (I) or a salt thereof is between 5 to 50 mg/kg, such as 10 to 40 mg/kg, 20 to 40 mg/kg, or 5 to 30 mg/kg. In some embodiments, the effective amount of a compound of formula (I) or a salt thereof is about 5 mg/kg, 7.5 mg/kg, 10 mg/kg, 12.5 mg/kg, 15 mg/kg, 17.5 mg/kg, 20 mg/kg, 22.5 mg/kg, 25 mg/kg, 27.5 mg/kg, 30 mg/kg, 32.5 mg/kg, 35 mg/kg, 37.5 mg/kg, 40 mg/kg, 42.5 mg/kg, 45 mg/kg, 47.5 mg/kg, or 50 mg/kg.

In at least one embodiment of the present disclosure, the pharmaceutical composition further comprises a pharmaceutically acceptable carrier. In some embodiments, the pharmaceutically acceptable carrier is selected from the group consisting of a filler, a binder, a preservative, a disintegrating agent, a lubricant, a suspending agent, a wetting agent, a flavoring agent, a thickening agent, an acid, a biocompatible solvent, a surfactant, a complexation agent, and any combination thereof, but the present disclosure is not limited thereto.

In at least one embodiment of the present disclosure, the pharmaceutical composition is in a form selected from the group consisting of a formulation to injection, dry powder, a tablet, an oral liquid, a wafer, a film, a lozenge, a capsule, a granule, a pill, a gel, a lotion, an ointment, an emulsifier, a paste, a cream, an eye drop, and a salve, but the present disclosure is not limited thereto.

In at least one embodiment of the present disclosure, the pharmaceutical composition is administered to the subject intravenously, subcutaneously, intradermally, orally, intrathecally, intraperitoneally, intranasally, intramuscularly, intrapleuraly, topically, or through nebulization, but the present disclosure is not limited thereto.

In another aspect, the present disclosure also provides a use of a compound of formula (I) below (i.e., FJU-C28) or a salt thereof in the manufacture of a medicament for preventing or treating an inflammatory pulmonary disease or disorder in a subject in need thereof:

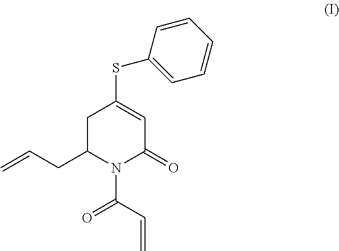 [SMILES: C=CCC1CC(Sc2ccccc2)=CC(=O)N1C(=O)C=C]

In the present disclosure, a 2-pyridone-based synthetic compound, FJU-C28, is provided to reduced neutrophil infiltration in the interstitium, lung damage and circulating levels of IL-6 and RANTES in a subject with an inflammatory pulmonary disease or disorder. FJU-C28 possesses anti-inflammatory activities to prevent endotoxin-induced lung function decrease and lung damages by down-regulating proinflammatory cytokines including IL-6 and RANTES via suppressing the JNK, p38 MAPK and NF-κB signaling pathways.

### BRIEF DESCRIPTION OF THE DRAWINGS

The present disclosure can be more fully understood by reading the following descriptions of the embodiments, with reference made to the accompanying drawings.

FIGS. 1A to 1E are graphs illustrating that effect of FJU-C28 on the activation of LPS-induced RAW264.7 macrophages.

FIGS. 2A to 2D are the graphs illustrating that inhibitory effects of FJU-C28 on the LPS-induced transcription of proinflammatory cytokines and inflammatory mediators.

FIG. 3 is the graph illustrating that array data of the expression profiles of cytokines in conditioned culture media from RAW264.7 macrophages treated with various compounds.

FIGS. 4A to 4D are graphs illustrating that FJU-C28 suppressed the LPS-induced expression of cytokines in RAW264.7 macrophages.

FIGS. 5A and 5B are graphs illustrating that the LPS-induced secretion of IL-6 and RANTES were mediated by various signaling pathways.

FIGS. 6A to 6D are graphs illustrating that effect of FJU-C28 on the LPS-induced phosphorylation of MAP kinases and NF-κB translocation.

FIG. 7 is the graph illustrating the effect of MAPK inhibitors and FJU-C28 on the activation of STAT3.

FIG. 8 is the graph illustrating the effect of FJU-C28 on STAT3 protein.

FIG. 9 is the graph illustrating a proposed model for FJU-C28 regulating proinflammatory responses via suppressing both LPS/TLR 4 and IL-6/STAT3 signaling.

FIGS. 10A to 10C are the graphs illustrating the effect of FJU-C28 on inhibiting STAT3 and smad3, TGF1β-induced alpha-SMA and fibronectin.

FIGS. 11A to 11F are the graphs illustrating the effect of FJU-C28 on preventing endotoxin-induced lung function decrease in mice with systemic inflammation.

FIGS. 12A and 12B are the graphs illustrating that FJU-C28 reduced lung damage and circulating levels of IL-6 and RANTES in mice with systemic inflammation.

### DETAILED DESCRIPTION OF THE EMBODIMENTS

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its scope, for different aspects and applications.

As used herein, the singular forms “a,” “an,” and “the” include plural referents, unless expressly and unequivocally limited to one referent. The term “or” is used interchangeably with the term “and/or,” unless the context clearly indicates otherwise.

As used herein, the term “comprising,” “comprises” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

As used herein, the terms “patient” and “subject” are used interchangeably. The term “subject” means a human or other animals. Examples of the subject include, but are not limited to, human, monkey, mice, rat, woodchuck, ferret, rabbit, hamster, cow, horse, pig, deer, dog, cat, fox, wolf, chicken, emu, ostrich, and fish. In some embodiments of the present disclosure, the subject is a mammal, e.g., a primate such as a human.

As used herein, the term “administering” or “administration” refers to the placement of an active agent into a subject by a method or route which results in at least partial localization of the active agent at a desired site to produce a desired effect. The active agent described herein may be administered by any appropriate route known in the art. For example, the pharmaceutical composition of the present disclosure is administered to the subject by oral administration.

The numeral ranges used herein are inclusive and combinable, any numeral value that falls within the numeral scope herein could be taken as a maximum or minimum value to derive the sub-ranges therefrom. For example, it should be understood that the numeral range “0.1 to 10 μM” comprises any sub-ranges between the minimum value of 0.1 μM to the maximum value of 10 μM, such as the sub-ranges from 0.1 μM to 5 μM, from 1.0 μM to 10 μM, from 0.5 μM to 8 μM and so on. In addition, a plurality of numeral values used herein can be optionally selected as maximum and minimum values to derive numerical ranges. For instance, the numerical ranges of 0.1 μM to 5 μM, 0.1 μM to 10 μM, and 5 μM to 10 μM can be derived from the numeral values of 0.1 μM, 5 μM, and 10 μM.

As used herein, the term “about” generally referring to the numerical value meant to encompass variations of +20%, +10%, +5%, +1%, +0.5%, or +0.1% from a given value or range. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations. Alternatively, the term “about” means within an acceptable standard error of the mean when considered by one of ordinary skill in the art. Unless otherwise expressly specified, all of the numerical ranges, amounts, values and percentages such as those for quantities of materials, durations of time periods, temperatures, operating conditions, ratios of amounts, and the likes disclosed herein should be understood as modified in all instances by the term “about.”

Numerous cytokines have been implicated in the pathogenesis of fibrosis, including, without limitation, transforming growth factor-β (TGF-β), tumor necrosis factor-α (TNF-α), platelet-derived growth factor (PDGF), insulin-like growth factor-1 (IGF-1), endothelin-1 (ET-1) and the interleukins, interleukin-1 (IL-1), interleukin-6 (IL-6), interleukin-8 (IL-8), and interleukin-17 (IL-17). Chemokine leukocyte chemoattractants, including the factor Regulated upon Activation in Normal T-cells, Expressed and Secreted (RANTES), are also thought to play an important role. Elevated levels of pro-inflammatory cytokines, such as Interleukin 8 (IL-8), as well as related downstream cell adhesion molecules (CAMs) such as intercellular adhesion molecule-1 (ICAM-1) and vascular cell adhesion molecule-1 (VCAM-1), matrix metalloproteinases such as matrix metalloproteinase-7 (MMP-7), and signaling molecules such as S100 calcium-binding protein A12 (S100A12, also known as calgranulin C), in the peripheral blood have been found to be associated with mortality, lung transplant-free survival, and disease progression in patients with idiopathic pulmonary fibrosis.

IL-6 is a major proinflammatory mediator that induces the acute-phase response, severe asthma and inflammatory pulmonary diseases. IL-6 is a major activator of signal transducer and activator of transcription 3 (STAT3) and blocks apoptosis in cells during the inflammatory process, keeping these cells alive in toxic environments. Several lines of evidence suggest that IL-6 is a pleiotropic cytokine during the transition from innate to acquired immunity to prevent increased tissue damage from the accumulation of neutrophil-secreted proteases and reactive oxygen species during inflammation. Studies have demonstrated that several signaling pathways, including the nuclear factor-κB (NF-κB) and mitogen activated protein kinase (MAPK) signaling pathways, are upregulated in animal models of acute lung injury. NF-κB plays a pivotal role in immune and inflammatory responses through the regulation of proinflammatory cytokines, adhesion molecules, chemokines, growth factors and inducible enzymes. Recently, reports have shown that p38 MAPK contributes to LPS-induced IL-6 secretion. Studies have shown that stimulating both the p38 MAPK and NF-κB signaling pathways can induce IL-6 gene expression and release.

RANTES (also called CCL5) is a C—C chemokine that plays an important role in recruiting leukocytes, including T lymphocytes, macrophages, eosinophils, and basophils, to inflammatory sites. Several infectious diseases caused by viruses, including dengue viruses, respiratory syncytial virus and influenza virus A, cause airway inflammation and significantly induce RANTES secretion and expression in humans and animal models. Also, SARS coronavirus (SARS-CoV) and respiratory syncytial virus (RSV) infection can induce high levels of IL-6 and RANTES (CCL5) in a cell model. High expression of RANTES after primary respiratory syncytial viral infection is associated with the exacerbation of airway disease; RSV-infected animals treated with anti-RANTES antibodies showed significant decreases in airway hyperreactivity (AHR). RANTES expression is associated with CD45-positive inflammatory cell infiltration, which causes pulmonary arterial hypertension. Several animal models of ARDS have shown elevated expression of RANTES induced by either LPS or caerulein, which lead to systemic inflammatory responses and distant lung injury. The treatment of caerulein-induced pancreatitis in mice with Met-RANTES can reduce lung damage. In addition, blocking the RANTES receptor CC-chemokine receptor type 5 may reduce and prevent lung damage in complement component 5a-induced acute lung injury. Hence, RANTES may be involved in various physiopathological processes and be a target for a new therapeutic strategy by interfering with the binding of this chemokine to its proteoglycan receptor.

Proinflammatory cytokines are important in cell signaling and promote systemic inflammation; cytokines are predominantly produced by activated macrophages and are involved in the upregulation of inflammatory reactions. Proinflammatory cytokines, such as TNFα and IL-6, modulate cell signaling and promote systemic inflammation. Recently, alternative anti-inflammatory medicinal compound Pirfenidone, a pyridone-related compound, was reported to inhibit the production of TNFα in vitro and in vivo and to prevent septic shock and subsequent mortality. The present disclosure demonstrated that the newly synthesized pyridine-related compound FJU-C28 could significantly reduce the LPS-induced expression of RANTES and IL-6.

In some embodiments, FJU-C28 is potentially advantageous for preventing inflammatory diseases by inhibiting the NF-κB and MAPK pathways. MAPKs and NF-κB play important roles in mediating extracellular signal transduction to the nucleus and activate the expression of inflammatory cytokines and mediators. In some embodiments, FJU-C28 may significantly suppress the expression of the proinflammatory cytokine IL-6 and the activation of STAT3 by regulating the NF-κB, p38 MAPK and JNK signaling pathways. NF-κB is an inactive form that is stabilized by the inhibitory protein IκBα in the cytoplasm and is activated in response to several stimuli, such as proinflammatory cytokines, infections, and physical stress. Activated NF-κB translocates from the cytoplasm to the nucleus and regulates the expression of proinflammatory and antiapoptotic genes. This pathway can also be amplified due to the inflammatory response by a positive NF-κB autoregulatory loop and increase the duration of chronic inflammation.

TNFα and IL-6 secretion, as well as neutrophil accumulation and protein leakage in the lungs of mice, was found to be dependent on p38 MAPK signaling. p38 MAPK is activated by a wide range of substrates, and the downstream activities attributed to these phosphorylation events are frequently cell type-specific, including inflammatory responses, cell differentiation, apoptosis, cytokine production and RNA splicing regulation. STAT3 phosphorylation is activated by MAPK, and these pathways play regulatory roles in the production of proinflammatory cytokines and downstream signaling events, leading to the synthesis of inflammatory mediators at the transcriptional and translational levels. Successfully suppressing IL-6 and inhibiting the activities of NF-κB and ERK, JNK, and p38 MAPK may have potential therapeutic value in inflammatory-mediated diseases, including the acute-phase response, chronic inflammation, autoimmunity, endothelial cell dysfunction and fibrogenesis.

In some embodiments, LPS-induced production of IL-6 is suppressed by FJU-C28 through inhibiting the activation of NF-κB, p38 and JNK signal pathways, and the activity of IL-6/STAT3 signaling also is inhibited via reducing the levels of STAT3 protein. It is suggested that the reduced levels of STAT3 protein may due to protein degradation. However, these data suggest that the synthetic compound FJU-C28 is a potential inflammatory therapeutic agent for inflammatory-mediated diseases mediated by IL-6/STAT3 signaling, including asthma and inflammatory lung diseases.

AMP-activated protein kinase (AMPK), a regulator of energy metabolism and autophagy, mediates energy homeostasis, including carbohydrate, lipid and protein metabolism. Recent studies have demonstrated that suppressing the activation of AMPK enhances LPS-induced inflammatory responses, including worsening the severity of ALI; conversely, reactivating AMPK exerts potent anti-inflammatory effects and attenuates LPS-induced acute lung injury in vitro and in vivo. Zhao et al. showed that RANTES/CCL5 activates autophagy through the AMPK pathway, and autophagy increases migration, which was confirmed by experiments with AMPK inhibitors. In some embodiments, it has been demonstrated that the secretion of IL-6 and RANTES was upregulated in the context of LPS-induced inflammation in vitro and in vivo. The current data suggest that RANTES may be involved in a proinflammatory response associated with hypercatabolism in patients with acute lung injury (ALI) and acute respiratory distress syndrome (ARDS). This finding suggested that IL-6 and RANTES could play important roles in energy metabolism in mice suffering from systemic inflammatory responses. In the present disclosure, it has been found that FJU-C28 was a highly potent compound that blocked the secretion of IL-6 and RANTES in LPS-activated macrophages and mice with endotoxemia. In some embodiments, the animal study also showed that treatment with FJU-C28 abrogated the LPS-induced decrease in lung function including vital capacity, lung compliance and forced vital capacity. This evidence strongly suggests that FJU-C28 is a highly promising therapeutic agent for the treatment of inflammatory lung injury that ameliorates declines in lung function due to virus-induced or endotoxin-induced systemic inflammatory responses by mediating RANTES and IL-6/STAT3 signaling.

### EXAMPLES

### Materials and Methods

The materials and methods used in the following Examples 1 to 7 were described in detail below. The materials used in the present disclosure but unannotated herein were commercially available.

### (1) Cell Culture

RAW264.7 cells (mouse monocyte/macrophage-like cells) were purchased from the Bioresource Collection and Research Center (Hsinchu, Taiwan). The cells were maintained in DMEM (HyClone, Logan, UT, USA) containing 10% fetal bovine serum (HyClone), MEM nonessential amino acids (HyClone), 100 mM sodium pyruvate (HyClone), and penicillin/streptomycin (HyClone). The cells were incubated at 37° C. in a humidified atmosphere of 5% CO2 and 95% air.

### (2) Chemicals

The FJU-Cs compound used in this study was synthesized at the Department of Chemistry at Fu-Jen Catholic University, Taiwan. Lipopolysaccharide (LPS) (Escherichia coli 0111:B4; Catalogue Number: L4391) was purchased from Sigma-Aldrich (Saint Louis, MO, USA). BAY11-7082 (NF-κB inhibitor), PD98059 (ERK1/2 inhibitor), SB203580 (p38 MAPK inhibitor) and SP600125 (JNK inhibitor) were purchased from Enzo Life Sciences (Farmingdale, NY, USA). Rapamycin (mTOR inhibitor) and Wortmannin (Phosphatidylinositol 3-kinase Inhibitor) was purchased from Abcam Biotechnology (Cambridge, UK). CLI-095 (TLR4 signaling inhibitor) was purchased from InvivoGen (San Diego, CA).

### (3) Cytokine Protein Array Analysis

Cytokine array analysis was performed according to the procedure recommended by the Raybio mouse cytokine antibody array 4 (RayBiotech, Inc. Peachtree Corners, GA). This cytokine protein array was used to simultaneously determine the relative levels of 40 different cytokines, chemokines, and acute-phase proteins in a single sample. One hundred microliters of cell culture medium was used for each sample. The signal intensity in the membrane of the cytokine protein array was measured by using ImageJ software and is presented as a heat map drawn by using MultiExperiment Viewer (MeV V.4.9.0) software.

### (4) Quantitative Real-Time PCR

RNA was isolated from cells using TRIzol reagent (Invitrogen, Carlsbad, CA, USA) as previously described.³² The concentration of the isolated RNA was measured using an Epoch microplate spectrophotometer (BioTek, Winooski, VT, USA). One microgram of RNA was reverse-transcribed to cDNA with random primers and an MMLV RT kit (Epicenter Biotechnologies, Madison, WI, USA). The mixture (2 μl) was added to PCR reagents to measure the target mRNA level with specific primers (Table 1). All real-time PCRs were performed in a volume of 20 μl containing 10 μl of Real-time PCR SYBR Green master mix (Toyobo, Osaka, Japan) using a PikoReal 96 Real-Time PCR System (Thermo Fisher Scientific Inc.), according to our previously described method.³³ The expression of β-actin was used as an internal control for RNA quantification.

TABLE 1
Real-time PCR Primers

| Gene | Forward Primer (5′) | Reverse Primer (3′) | Size (bp) |
|---|---|---|---|
| iNOS | GAAGAAAACCCCTTGTGCTG | GTCGATGTCACATGCAGCTT | 138 |
| COX2 | GATGTTTGCATTCTTTGCCC | GGCGCAGTTTATGTTGTCTG | 149 |
| IL-1β | CGCAGCAGCACATCAACAAGAGC | TGTCCTCATCCTGGAAGGTCCACG | 111 |
| IL-6 | CACAAGTCCGGAGAGGAGAC | CAGAATTGCCATTGCACAAC | 141 |
| β-actin | GATTACTGCTCTGGCTCCTAGC | GACTCATCGTACTCCTGCTTGC | 147 |

### (5) Enzyme-Linked Immunosorbent Assay (ELISA)

RANTES (RayBiotech), IL-1β and IL-6 (eBioscience, San Diego, CA, USA) concentrations in cell culture media and mouse serum were measured using ELISA kits according to the manufacturer's instructions. The plates were measured at 450 nmol/L using an Epoch microplate spectrophotometer (BioTek Instruments, Winooski, VT, USA). The concentrations of RANTES, IL-1β and IL-6 in the samples were determined by standard curves.

### (6) Western Blotting

In brief, cell lysate was separated by 10% SDS-PAGE and transferred to a PVDF membrane (Hybond™-P, Amersham, Piscataway, NJ, USA). The blots were probed with anti-p38 (Catalogue Number: 8690P), anti-p-p38 (Thr180/Tyr182; Catalogue Number: 4511P), anti-ERK44/42 (Catalogue Number: 4695P), anti-p-ERK44/42 (Thr202/Tyr204; Catalogue Number: 4370P), anti-JNK (Catalogue Number: 9258P), anti-p-JNK (Thr183/Tyr185; Catalogue Number: 4668P), anti-p65 (Catalogue Number: 8242S), anti-STAT3 (Catalogue Number: 12640S), anti-p-STAT3 (Tyr705; Catalogue Number: 9145S) and anti-RANTES (Catalogue Number: 2989S) antibodies were provided by Cell Signaling Technology, Inc. (Danvers, MA, USA). Anti-Lamin A/C (Catalogue Number: 101127) antibodies were obtained from GeneTex, Inc. (San Antonio, TX, USA). Anti-β-Actin (Catalogue Number: SC-47778) and anti-COX2 (Catalogue Number: SC-1746) antibody was obtained from Santa Cruz Biotechnology, Inc. (Dallas, Texas, USA). The anti-iNOS (Catalog Number: 610329) antibody was obtained from BD transduction Lab. (San Jose, CA, USA). Bound antibodies were visualized by electrochemical luminescence staining (Western Lighting Plus ECL; PerkinElmer, Wellesley, MA, USA) with autoradiography using FUJI Medical X-ray film (Fuji Corporation, Kofu, Yamanashi, Japan) or a MultiGel-21 multifunctional imaging system (TOPBIO, New Taipei, Taiwan). The intensity of the western blot bands was quantified by ImageJ software. The quantitative immunoblot data were normalized to the internal control protein and are expressed as the relative ratio of the treatment group to the control. The data represent the mean±S.D. of at least three independent experiments.

### (7) Analysis of Cell Viability

RAW264.7 macrophages were pretreated with FJU-Cs (0 to 10 μM) as indicated for 30 min and were then stimulated with/without LPS (100 ng/ml) for 24 h. The remaining cells were evaluated by a 3-(4,5-dimethylthiazol-2-yl)-2,5-diphenyl tetrazolium bromide (MTT) assay. Cells containing formazan crystals were dissolved in DMSO (Merck, Darmstadt, Germany) and quantified at a wavelength of 595 nm using a spectrophotometer (BioTek Instruments, Inc. Winooski, Vermont, USA). Each experiment was repeated at least 3 times.

### (8) Animal Model

Ten-week-old male C57BL/6 mice from BioLasco Taiwan Co., Ltd. (Taipei, Taiwan) were used in the present study. The mice were maintained under standard laboratory conditions as described in a previous study.³⁴ All animal procedures were approved by the Institutional Animal Care and Use Committee of Fu Jen Catholic University (IACUC approval number: A10508). We also confirmed that all experiments were performed in compliance with the ARRIVE guidelines. All surgeries were performed under anesthesia, and all efforts were made to minimize the pain and discomfort of the animals. The mice were randomly assigned to three groups: control (n=7), LPS (n=7) and LPS plus C28 (n=8). The mice were injected with/without FJU-C28 (5 mg/kg) dissolved in DMSO/PBS buffer, and after one hour the mice were stimulated with an intraperitoneal injection of LPS (7.5 mg/kg) in PBS buffer. At 24 hours after LPS stimulation, the mice were anesthetized i.p. with a mixture of ketamine (100 mg/kg) (Pfizer, New York, US) and xylazine (10 mg/kg) (Bayer, Leverkusen, Germany), endotracheally intubated with an airway catheter and connected to a forced pulmonary maneuver system (Buxco Research System; Buxco Electronics, Wilmington, NC). The detailed procedure was performed as described in a previous study.³⁴ The lung function values, including C chord (lung compliance), IC (inspiratory capacity), VC (vital capacity), FEV100 (forced expiratory volume at 100 ms) and FVC (forced vital capacity), were measured using a Buxco Research System. After the lung function assays, the experimental mice were sacrificed, and blood was collected by cardiac puncture. The lung lobes were inflated with 4% buffered paraformaldehyde via a catheter. Slides of lung specimens at a thickness of 5 μm were stained with hematoxylin and eosin for light microscopic analysis.

### (9) Statistical Analysis

The data for triplicate experiments are expressed as the means and standard errors. All statistical analyses were performed by one-way ANOVA, followed by Tukey's multiple comparison post hoc test. A value of p<0.05 was considered to be significant.

### Example 1: Effect of FJU-C28 on the Activation of LPS-Induced RAW264.7 Macrophages

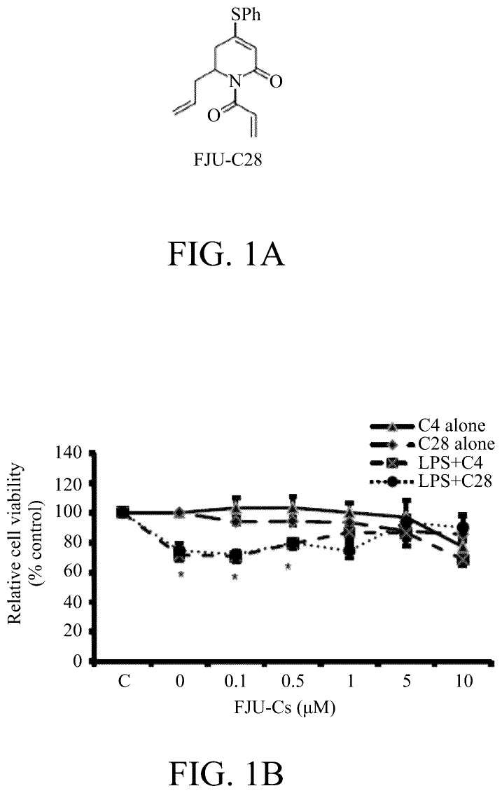

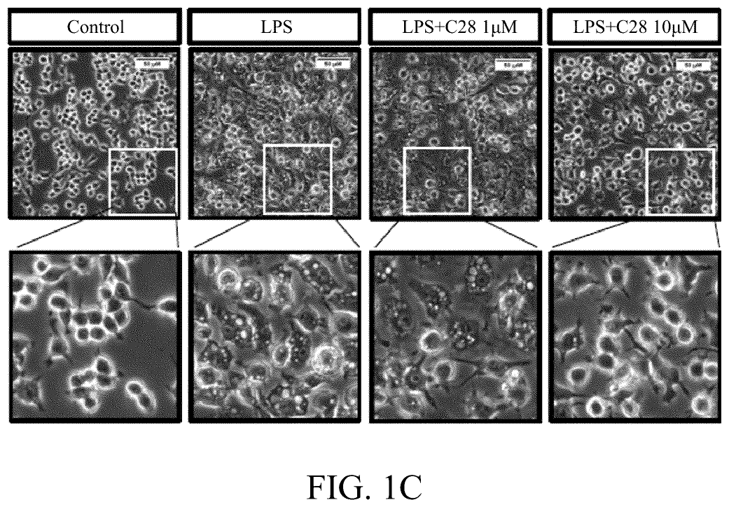

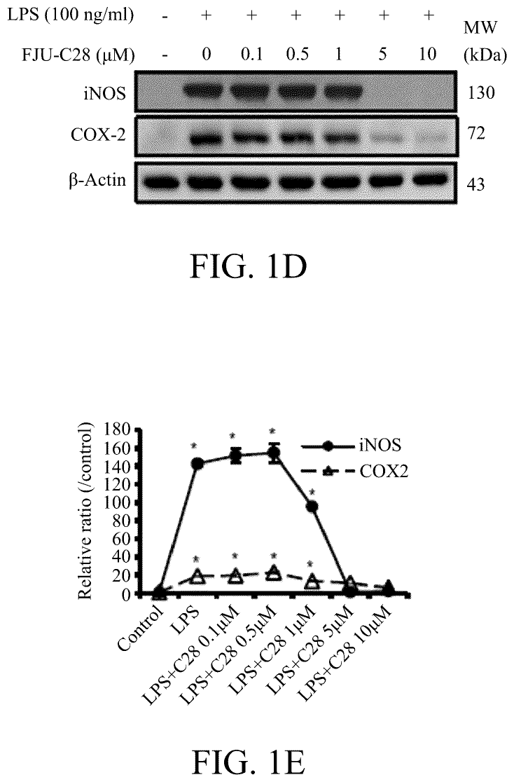

To compare the anti-inflammatory effects of the newly synthesized compound FJU-C28 (FIG. 1A) on LPS-activated murine macrophages to those of the parent compound FJU-C4, RAW264.7 macrophages were pretreated with various concentrations of FJU-Cs for 30 min and then stimulated with or without LPS for 24 hours. FJU-C28 protected RAW264.7 macrophages from LPS-induced cell death and exhibited less cytotoxicity than 10 μM FJU-C4 (FIG. 1B). LPS stimulation changed the shape of macrophages, resulting in dendritic-like cells with multiple vacuoles in the cytoplasm, whereas the untreated cells were round and small. FJU-C28 dramatically inhibited these changes in morphology in a concentration-dependent manner, and the morphology was similar to that of untreated cells (FIG. 1C). This effect was consistent with the findings of the cytotoxicity assay. This result indicated that FJU-C28 could suppress the inflammatory response induced by LPS in RAW264.7 macrophages. In addition, FJU-C28 suppressed the expression of iNOS and COX2 at doses higher than 5 μM (FIG. 1D). The quantitative immunoblot data are shown in FIG. 1E.

### Example 2: Inhibitory Effects of FJU-C28 on the Transcriptional Regulation of Inflammatory Mediators and Proinflammatory Cytokines

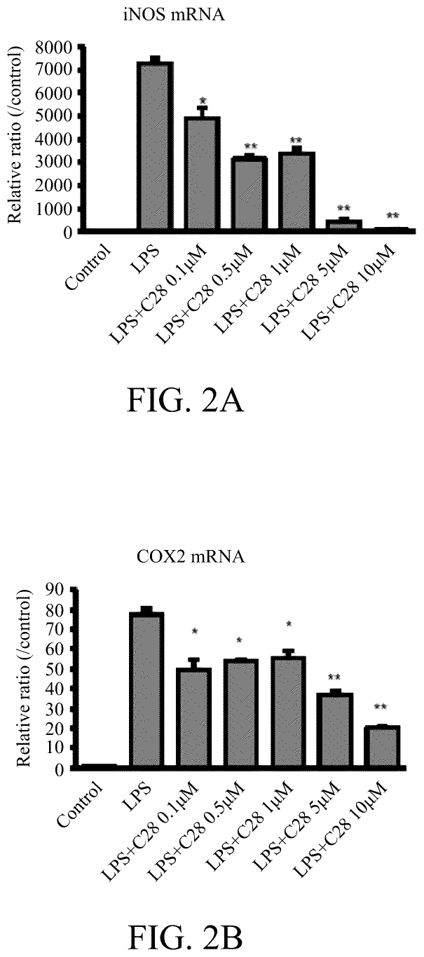

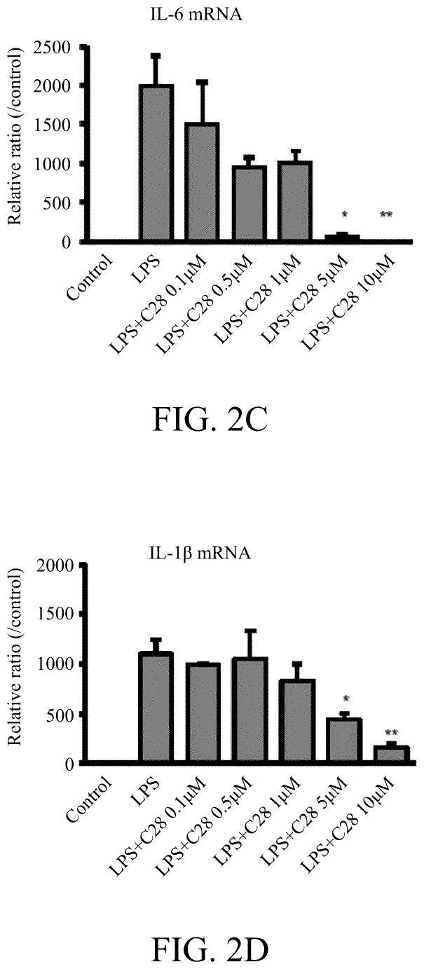

The effects of FJU-C28 on the gene expression of proinflammatory cytokines and late inflammatory mediators in macrophages were analyzed by using quantitative real-time RT-PCR (FIG. 2). The results showed that the mRNA levels of iNOS and COX2 were downregulated in a concentration-dependent manner. The mRNA levels of the proinflammatory cytokine IL-6 and IL-1β were concentration-dependently decreased when the concentration of FJU-C28 was less than 10 μM. FJU-C28 noticeably inhibited the gene expression of IL-6 and iNOS when the dose was higher than 5 μM. These results indicated that FJU-C28 significantly reduced the transcription of the proinflammatory cytokine including IL-6 and IL-1β in LPS-induced RAW264.7 macrophages.

### Example 3: Cytokine Expression Profile in Various Conditioned Media

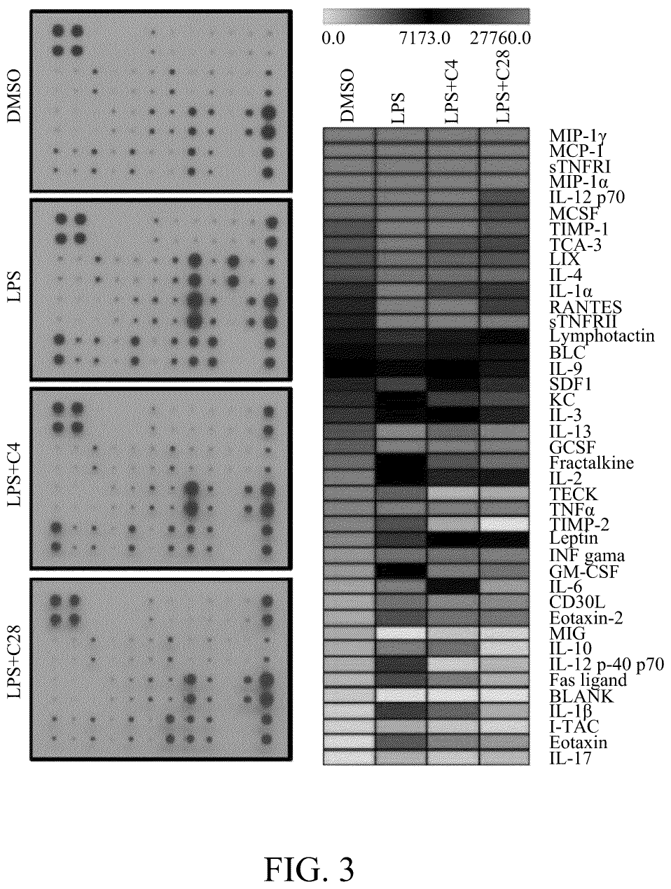

To distinguish the effect of various FJU-Cs on LPS-induced inflammation, the cell culture media of RAW264.7 macrophages treated with various conditions were harvested and analyzed by using a mouse cytokine antibody array (left panel of FIG. 3). The signal intensities on the array membranes were quantified by densitometry, and the changes in different cytokines are represented as a heat map (right panel of FIG. 3). The results showed that several cytokines, including IL-10, IL-6, GCSF, eotaxin, TNFα, IL-17, IL-1β, leptin, sTNF RII, and RANTES, were enhanced by LPS stimulation by at least 5-fold compared to those in the culture media of untreated cells. Moreover, the secretion of the RANTES, TIMP1, IL-6 and IL-10 cytokines was dramatically suppressed by FJU-C28 treatment. Although LPS-induced secretion of TIMP1, IL-6 and IL-10 was also suppressed by FJU-C4 treatment, the expression of RANTES was not changed by FJU-C4 treatment (Table 2).

TABLE 2
Protein array analysis

| | | Protein array, fold change (mean ± S.E.) | | |
|---|---|---|---|---|
| | | LPS/ | LPS + C4/ | LPS + C28/ |
| Symbol | Control | control | control | control |
| IL-10 | 1.0 | 63.6 ± 1.7 | 2.2 ± 0.6 | 0.3 ± 0.2 |
| IL-6 | 1.0 | 53.9 ± 2.9 | 5.5 ± 0.0 | 1.1 ± 0.2 |
| GCSF | 1.0 | 22.3 ± 0.7 | 18.8 ± 1.0 | 20.1 ± 1.9 |
| Eotaxin | 1.0 | 20.3 ± 0.1 | 13.5 ± 2.9 | 10.3 ± 6.1 |
| TNFα | 1.0 | 12.3 ± 1.0 | 11.8 ± 0.9 | 21.0 ± 0.8 |
| IL-17 | 1.0 | 9.8 ± 1.3 | 4.6 ± 0.8 | 7.8 ± 5.3 |
| IL-1B | 1.0 | 8.5 ± 0.4 | 6.3 ± 0.2 | 2.6 ± 0.0 |
| Leptin | 1.0 | 6.3 ± 0.9 | 2.7 ± 0.2 | 3.2 ± 0.8 |
| STNFRII | 1.0 | 5.4 ± 0.3 | 2.2 ± 0.1 | 2.1 ± 0.1 |
| RANTES | 1.0 | 5.2 ± 0.4 | 5.0 ± 0.5 | 1.3 ± 0.2 |
| IL-12 p-40 p70 | 1.0 | 3.7 ± 0.9 | 0.4 ± 0.9 | 0.9 ± 0.1 |
| GM-CSF | 1.0 | 3.5 ± 0.3 | 1.4 ± 0.0 | 1.5 ± 0.4 |
| Fas ligand | 1.0 | 3.5 ± 0.1 | 2.3 ± 0.9 | 1.1 ± 0.8 |
| I-TAC | 1.0 | 3.1 ± 0.6 | 1.5 ± 0.2 | 0.8 ± 0.3 |
| SDF1 | 1.0 | 3.1 ± 0.2 | 1.5 ± 0.3 | 0.9 ± 0.2 |
| Eotaxin-2 | 1.0 | 2.8 ± 0.3 | 2.1 ± 0.1 | 2.0 ± 1.0 |
| TIMP-1 | 1.0 | 2.8 ± 0.2 | 1.9 ± 0.2 | 1.1 ± 0.1 |
| MCP-1 | 1.0 | 2.7 ± 0.1 | 2.5 ± 0.2 | 1.7 ± 0.2 |
| IL-2 | 1.0 | 2.6 ± 0.1 | 2.0 ± 0.1 | 2.1 ± 0.2 |
| Fractalkine | 1.0 | 2.1 ± 0.1 | 1.2 ± 0.2 | 1.0 ± 0.6 |
| CD30 L | 1.0 | 2.0 ± 0.4 | 1.6 ± 0.1 | 1.6 ± 0.9 |
| IL-1α | 1.0 | 1.7 ± 0.1 | 1.3 ± 0.1 | 1.1 ± 0.1 |
| IL-3 | 1.0 | 1.7 ± 0.1 | 1.5 ± 0.1 | 1.1 ± 0.2 |
| TIMP-2 | 1.0 | 1.6 ± 0.4 | 0.6 ± 0.0 | 0.0 ± 0.0 |
| INF gamma | 1.0 | 1.6 ± 0.2 | 1.5 ± 0.2 | 1.4 ± 0.2 |
| STNFRI | 1.0 | 1.5 ± 0.1 | 1.1 ± 0.1 | 1.0 ± 0.0 |
| BLC | 1.0 | 1.4 ± 0.1 | 1.3 ± 0.1 | 1.3 ± 0.1 |
| Lymphotactin | 1.0 | 1.4 ± 0.1 | 1.0 ± 0.0 | 0.7 ± 0.2 |
| MCSF | 1.0 | 1.4 ± 0.1 | 1.0 ± 0.2 | 0.8 ± 0.1 |
| TECK | 1.0 | 1.3 ± 0.2 | 0.5 ± 0.3 | 0.5 ± 0.3 |
| MIP-1α | 1.0 | 1.3 ± 0.1 | 1.2 ± 0.1 | 1.0 ± 0.2 |
| TCA-3 | 1.0 | 1.3 ± 0.1 | 1.0 ± 0.1 | 0.9 ± 0.1 |
| KC | 1.0 | 1.3 ± 0.0 | 0.9 ± 0.0 | 0.8 ± 0.4 |
| IL-4 | 1.0 | 1.3 ± 0.0 | 1.2 ± 0.1 | 1.2 ± 0.2 |
| IL-12 p70 | 1.0 | 1.2 ± 0.1 | 1.0 ± 0.1 | 0.8 ± 0.2 |
| LIX | 1.0 | 1.2 ± 0.1 | 1.1 ± 0.1 | 1.0 ± 0.2 |
| IL-9 | 1.0 | 1.2 ± 0.2 | 1.0 ± 0.2 | 0.9 ± 0.3 |
| MIP-1γ | 1.0 | 1.0 ± 0.0 | 1.0 ± 0.0 | 1.2 ± 0.2 |
| IL-13 | 1.0 | 0.6 ± 0.0 | 0.8 ± 0.0 | 0.6 ± 0.0 |

### Example 4: FJU-C28 Suppressed the LPS-Induced Expression of RANTES and IL-6

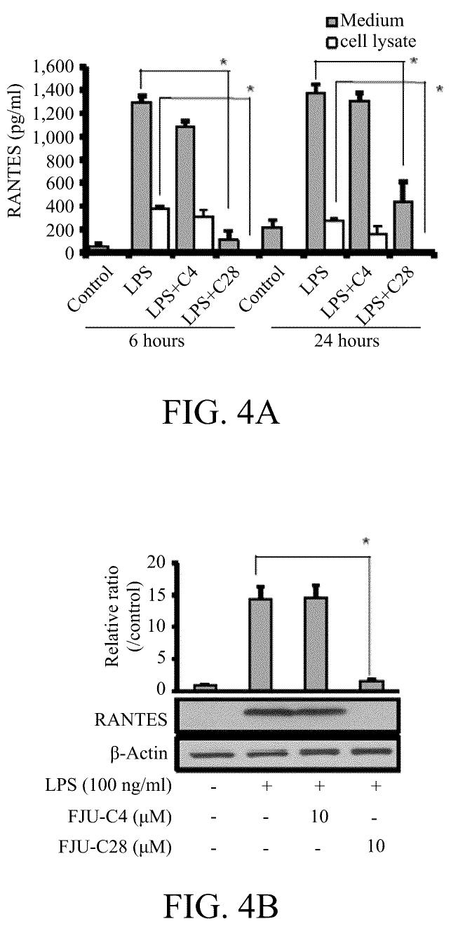

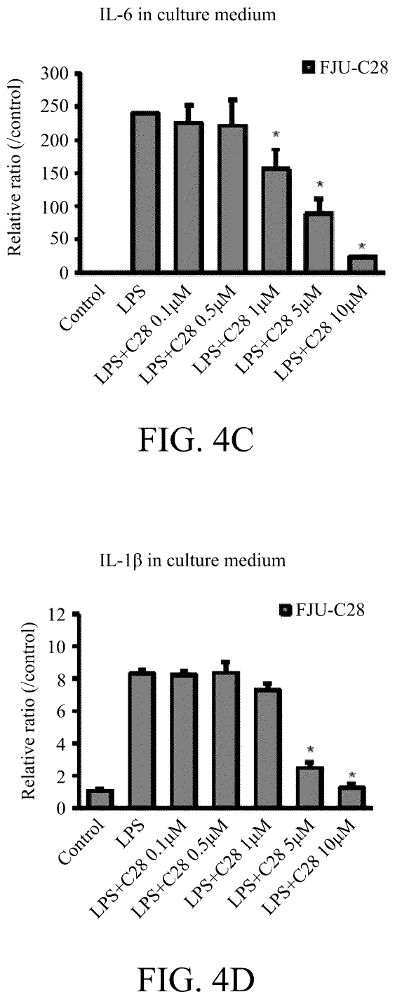

To confirm whether FJU-C28 can suppress the expression of RANTES in RAW264.7 macrophages, the cell culture media and cell lysates of RAW264.7 macrophages treated with various conditions were harvested for ELISA and western blot analysis. The ELISA results showed that the expression of RANTES was enhanced in cell culture media and cell lysates of LPS-treated cells; FJU-C28 dramatically suppressed the LPS-induced expression of RANTES, but FJU-C4 was unable to reduce the LPS-induced expression of RANTES at 6 hours or 24 hours (FIG. 4A). In addition, western blot analysis also confirmed that FJU-C28 dramatically suppressed the LPS-induced protein expression of RANTES at 6 hours, but FJU-C4 did not reduce the protein expression of RANTES in treated cells (FIG. 4B). The secretion of proinflammatory cytokines such as IL-6 and IL-1β in cell culture media was also analyzed by ELISA (FIGS. 4C and 4D). The results showed that FJU-C28 dramatically reduced the production of RANTES, IL-6 and IL-1β in LPS-treated macrophages.

### Example 5: Effects of FJU-C28 on LPS-Induced MAP Kinase Phosphorylation and NF-κB Translocation

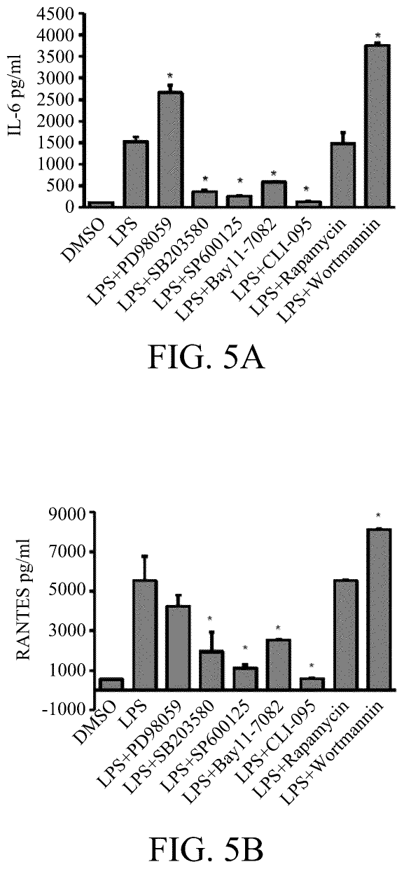

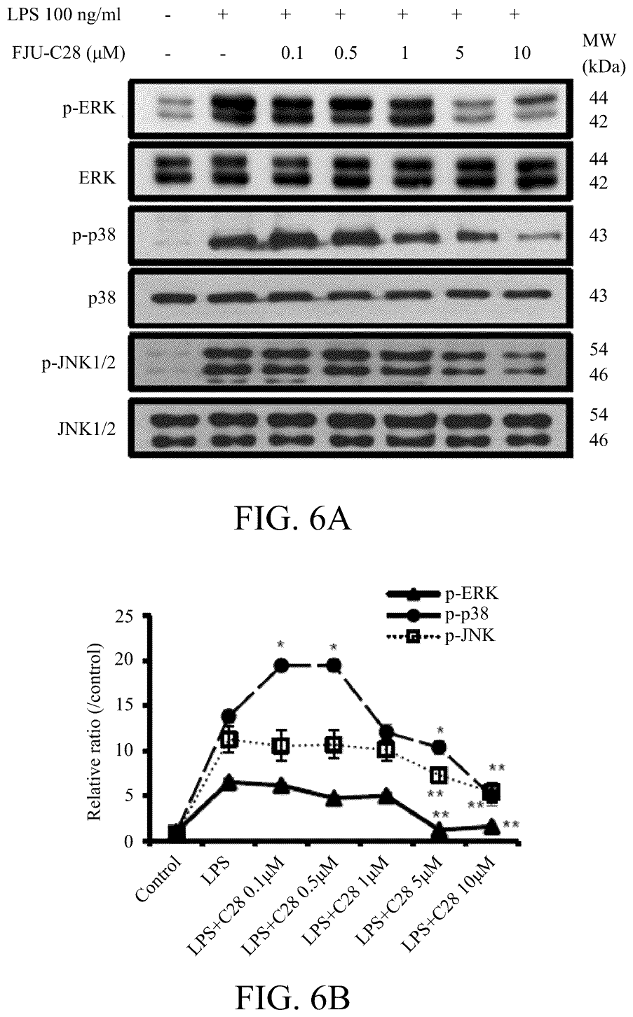

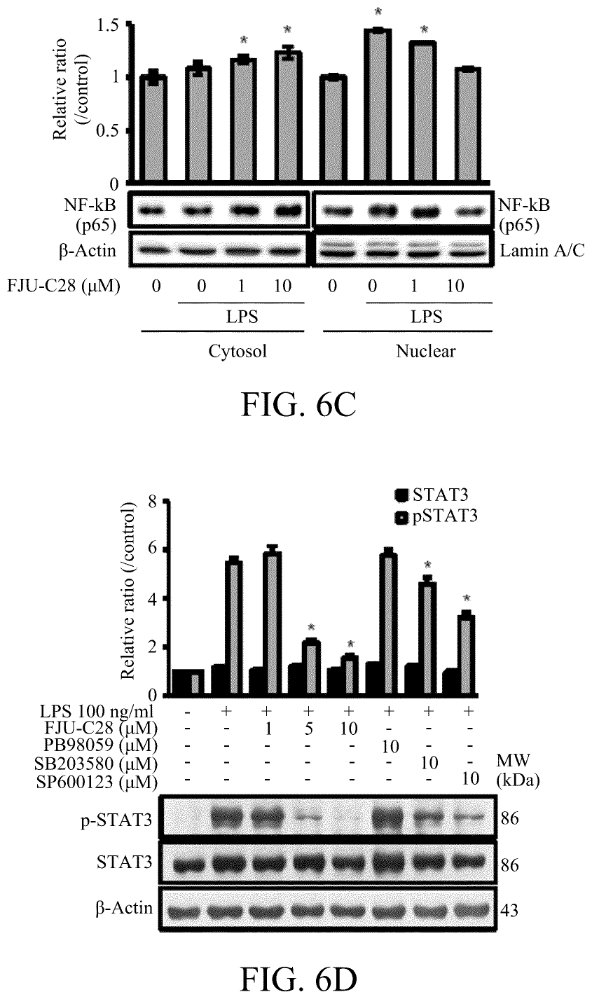

For identifying the potential signaling pathways which regulate the secretion of IL-6 and RANTES induced by LPS stimulation, RAW264.7 macrophages were pretreated with various kinase inhibitors for 30 min and then stimulated with/without 100 ng/ml LPS for 24 h; the cell culture media was harvested for ELISA assay. The results showed that LPS-induced expression of IL-6 and RANTES were suppressed by CLI-095 (TLR4 inhibitor), BAY11-7082 (IkB-α Inhibitor), SB203580 (p38 MAPK inhibitor) and SP600125 (JNK inhibitor), but not by PD98059 (ERK inhibitor), Rapamycin (mTOR inhibitor) and Wortmannin (Phosphatidylinositol 3-kinase Inhibitor) (FIGS. 5A and 5B). It indicated that LPS stimulate TLR4 signaling to induce the expression of IL-6 and RANTES through activation of NF-κB, p38 and JNK signaling pathways but not ERK, PI3K or mTOR signaling pathways. We further investigated the potential inhibitory effect of FJU-C28 on the activation of MAPKs in LPS-stimulated macrophages. Treatment with 5 μM and 10 μM FJU-C28 dramatically inhibited LPS-induced levels of p-ERK, p-p38 and p-JNK MAP kinase in macrophages. (FIG. 6A). The quantitative immunoblot data are shown in FIG. 6B. To investigate whether FJU-C28 could mediate the transcriptional activity of NF-κB (p65), the translocation of NF-κB p65 was examined in LPS-stimulated macrophages. The results showed that the level of NF-κB p65 in the nuclear extract was markedly increased in LPS-stimulated cells compared to that in untreated cells. As the dose of FJU-C28 administered to LPS-stimulated RAW264.7 macrophages was increased, nuclear NF-κB (p65) was decreased; conversely, NF-κB (p65) accumulated in the cytosolic extract (FIG. 6C). This result indicated that FJU-C28 could suppress NF-κB transcriptional activity by blocking LPS-induced NF-κB translocation from the cytosol to the nucleus in a concentration-dependent manner.

### Example 6: Effects of FJU-C28 on IL-6/STAT3 Signaling

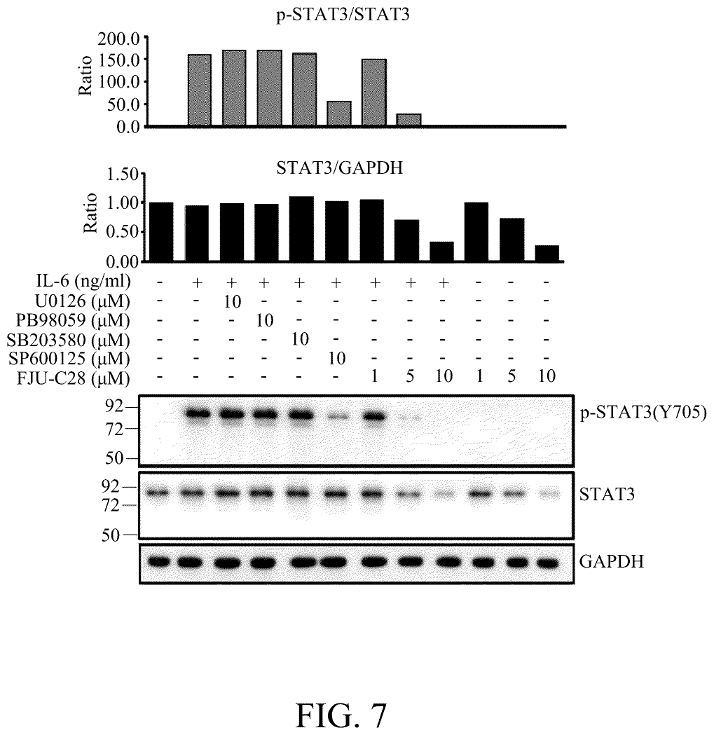

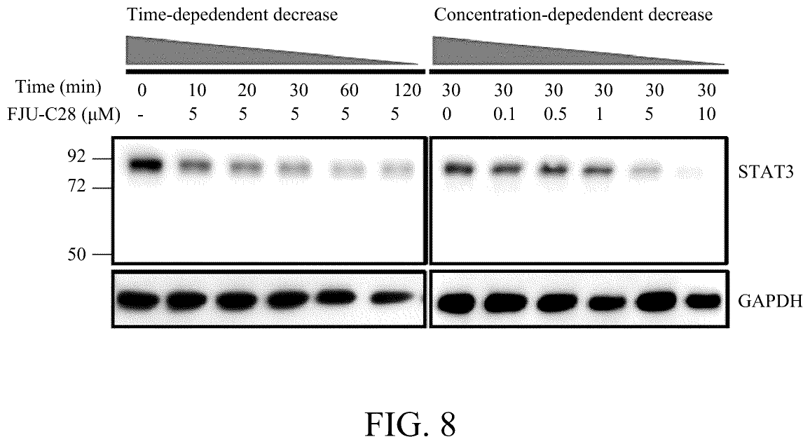

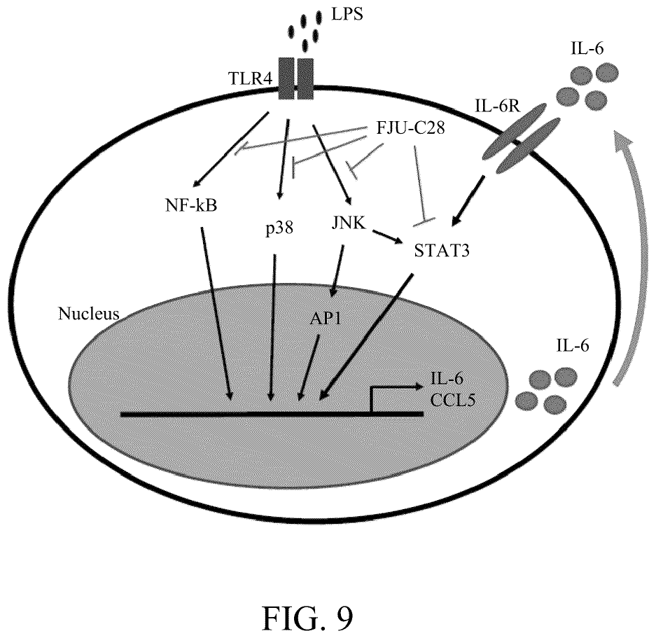

To investigate the suppressive effect of FJU-C28 on IL-6/STAT3 signaling in LPS-induced macrophage inflammation, LPS-stimulated cells were treated with FJU-C28 or various MAPK inhibitors, and cell lysates were analyzed by western blot analysis. The results showed that FJU-C28 concentration-dependently inhibited the phosphorylation of STAT3 in LPS-induced RAW264.7 macrophages. In addition, MAPK inhibitors, including SB203580 (p38 MAPK inhibitor) and SP600125 (JNK inhibitor), inhibited the LPS-induced phosphorylation of STAT3 in RAW264.7 macrophages, but PD98059 (ERK inhibitor) slightly suppressed STAT3 phosphorylation. Taken together, these findings show that FJU-C28 plays an important role in suppressing the LPS-induced activation of IL-6/STAT3 signaling by suppressing the activation of JNK and p38 MAPK which mediates the LPS-induced expression of IL-6 in RAW264.7 macrophages (FIG. 6D). In addition, the suppressive effect of SP600125 was better than SB203580 because SP600125 also contributed the inhibitory effect on the phosphorylation of STAT3 induced by IL-6 stimulation (FIG. 7). Interestingly, FJU-C28 can dramatically suppress the IL-6/STAT3 signaling induced by LPS through not only inactivating p38 and JNK but also reducing the levels of STAT3 protein (FIG. 8). The proposed mechanism of action regarding FJU-C28 suppressing the IL-6/STAT3 was illustrated on FIG. 9.

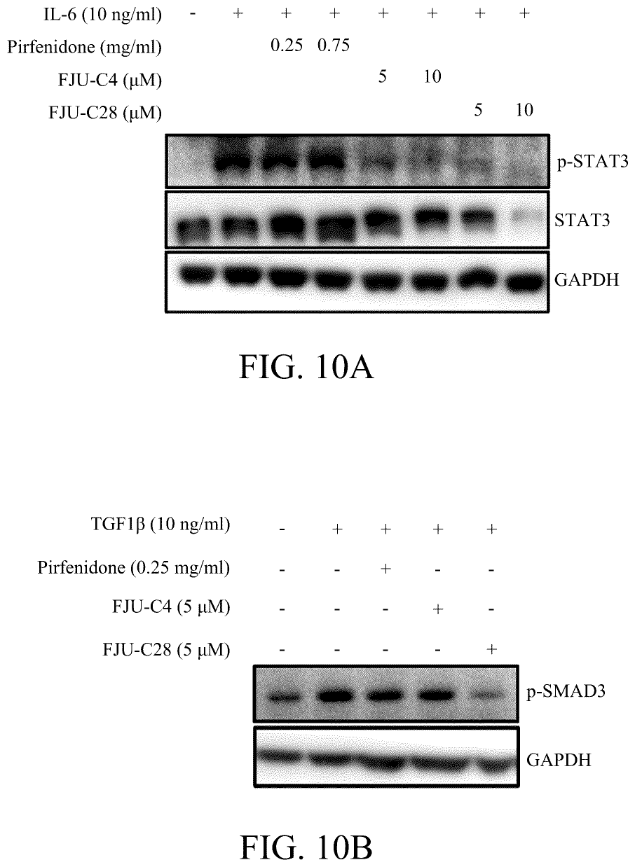

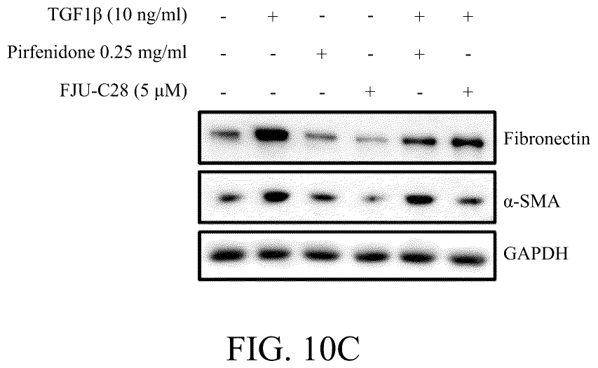

Further, a comparison between FJU-C28 and FDA-approved drug Pirfenidone for effects on STAT3 inhibition, smad3 activation, TGF1β-induced alpha-SMA and fibronectin expression are conducted. The results are shown in the quantitative immunoblot data in FIGS. 10A, 10B and 10C. According to FIGS. 10A, 10B and 10C, the effects on inhibiting STAT3, smad3, TGF1β-induced alpha-SMA and fibronectin expression of FJU-C28 is more significant than that of Pirfenidone.

### Example 7: Effect of FJU-C28 on Lung Function in Mice with LPS-Induced Systemic Inflammation

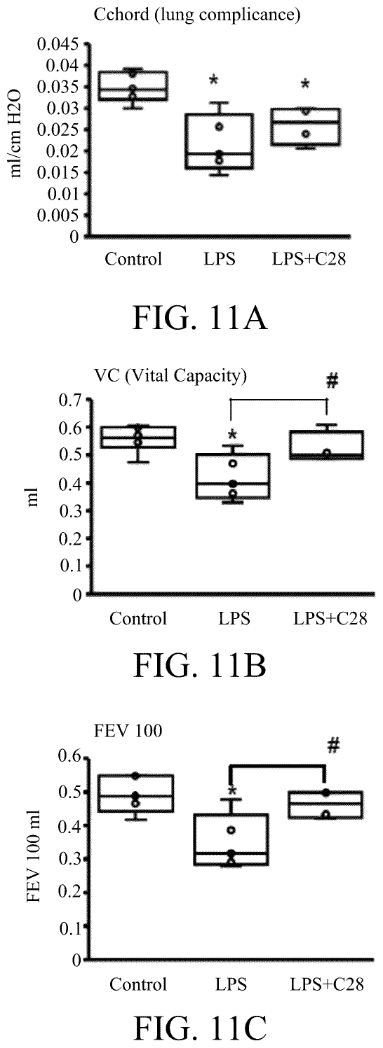

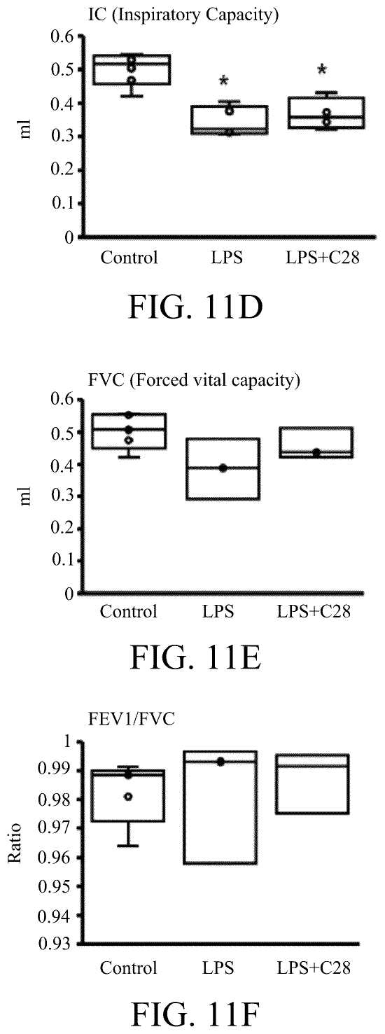

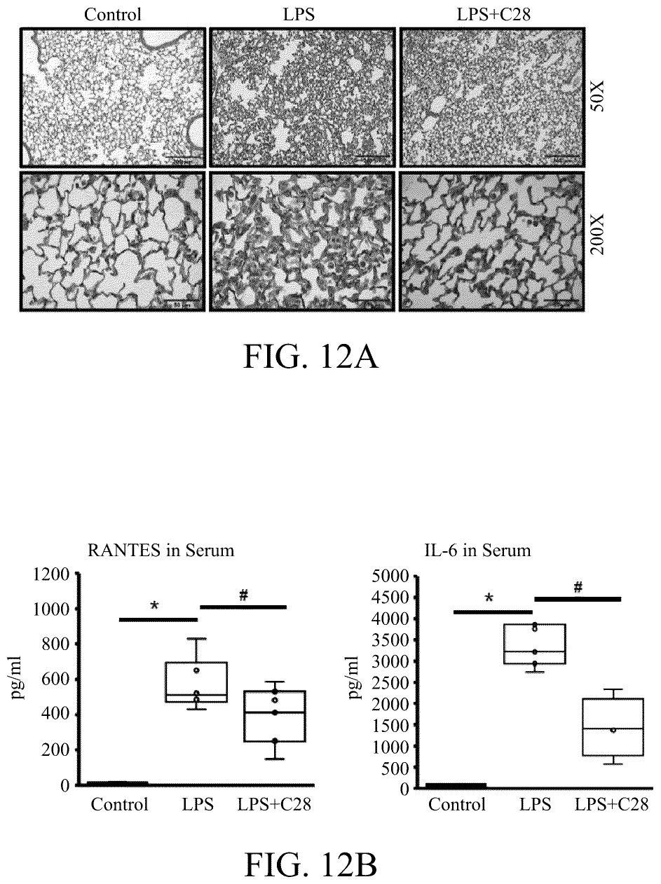

To evaluate the protective effect of FJU-C28 on lung function in mice with systemic inflammation induced by endotoxin, male C57BL/6 mice were administered LPS (7.5 mg/kg) and treated with/without FJU-C28 (5 mg/kg) for 24 hours; subsequently, lung function parameters were measured using a Buxco pulmonary function test system. The results showed that compared with normal control mice, mice with LPS-induced systemic inflammation had significantly decreased lung inspiratory capacity (IC), vital capacity (VC), lung compliance (C chord), forced expiratory volume at 100 ms (FEV100), and forced vital capacity (FVC). However, treatment with FJU-C28 restored the LPS-induced decreases in lung function including VC (p<0.05), C chord, FEV100 (p<0.05), and FVC (p<0.05), compared to those of LPS-treated mice (FIG. 11). To confirm the status of LPS-induced lung injury in mice, histological examination of lung specimens was performed by H&E staining. The results demonstrated that neutrophils infiltrated and accumulated in the interstitium of the lungs, and the alveolar structure was destroyed and became thickened and irregular in mice in the LPS stimulation group compared to mice in the control group. Treatment with FJU-C28 reduced the neutrophil infiltration in the interstitium and sustained most of the alveolar structure in mouse lung tissue in the LPS+FJU-C28 treatment group compared to the LPS stimulation group (FIG. 12A). To ascertain the correlation between target cytokines and lung injury, serum cytokines were measured by ELISA. The results showed that the circulating levels of IL-6 and RANTES were significantly elevated in mice with LPS-induced systemic inflammation compared to control mice. The secretion of RANTES and IL-6 was significantly suppressed by FJU-C28 treatment (FIG. 12B). This finding indicated that FJU-C28 could attenuate lung injury by suppressing the secretion of proinflammatory cytokines, including IL-6 and RANTES, in LPS-induced systemic inflammation.

The present disclosure unexpectedly finds that FJU-C28 possesses anti-inflammatory activities to prevent endotoxin-induced lung function decrease and lung damages by down-regulating proinflammatory cytokines including IL-6 and RANTES via suppressing the JNK, p38 MAPK and NF-κB signaling pathways. The present disclosure provides additional insight into the mechanism and new opportunities for therapeutic intervention against lung inflammatory disease.

While some of the embodiments of the present disclosure have been described in detail above, it is possible for those of ordinary skill in the art to make various modifications and changes to the embodiments shown without substantially departing from the teaching and advantages of the present disclosure. Such modifications and changes are encompassed in the scope of the present disclosure as set forth in the appended claims.

1. A method for preventing or treating an inflammatory pulmonary disease or disorder, comprising administering a pharmaceutical composition to a subject in need thereof, wherein the pharmaceutical composition comprises an effective amount of a compound of formula (I) below or a salt thereof:

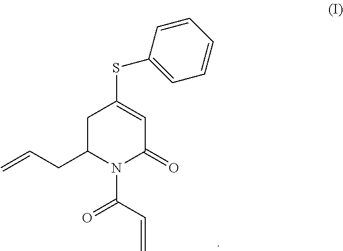 [SMILES: C=CCC1CC(Sc2ccccc2)=CC(=O)N1C(=O)C=C]

2. The method of claim 1, wherein the inflammatory pulmonary disease is acute respiratory distress syndrome or lung fibrosis.

3. The method of claim 1, wherein the compound of formula (I) or a salt thereof is used to suppress mRNA or protein expression of iNOS in the subject.

4. The method of claim 1, wherein the compound of formula (I) or a salt thereof is used to suppress mRNA or protein expression of COX2 in the subject.

5. The method of claim 1, wherein the compound of formula (I) or a salt thereof is used to suppress mRNA or protein expression of a proinflammatory cytokine in the subject.

6. The method of claim 5, wherein the proinflammatory cytokine is selected from a group consisting of IL-10, IL-6, GCSF, Eotaxin, TNFα, IL-17, IL-1β, Leptin, sTNFRII, RANTES, IL-12 p-40 p70, GM-CSF, Fas ligand, I-TAC, SDF1, Eotaxin-2, TIMP-1, MCP-1, IL-2, Fractalkine, CD30 L, IL-1α, IL-3, TIMP-2, INF gamma, sTNFRI, BLC, Lymphotactin, MCSF, TECK, MIP-1α, TCA-3, KC, IL-4, IL-12 p70, LIX, IL-9, MIP-1γ, IL-13, MIG, and a combination thereof.

7. The method of claim 6, wherein the proinflammatory cytokine is selected from a group consisting of RANTES, TIMP1, IL-6, IL-10, and a combination thereof.

8. The method of claim 7, wherein the proinflammatory cytokine is RANTES or IL-6.

9. The method of claim 1, wherein the effective amount of a compound of formula (I) or a salt thereof is between 0.1 to 10 μM.

10. The method of claim 1, wherein the effective amount of a compound of formula (I) or a salt thereof is between 5 to 50 mg/kg.

11-20. (canceled)
